(19) United States
(12) Patent Application Publication
(10) Pub. No.: US 2010/0185097 A1
(43) Pub. Date: Jul. 22, 2010

(54) ACOUSTIC OFFSET FOR TRANDUCER

(75) Inventor: Christoper S. Hall, Hopewell Junction, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/665,586

(22) PCT Filed: Jun. 20, 2008
(86) PCT No.: PCT/IB08/52450

§ 371 Date: Dec. 18, 2009

Related U.S. Application Data
(60) Provisional application No. 60/945,599, filed on Jun. 22, 2007.

(51) Int. Cl. A61B 8/00

(52) U.S. Cl. 600/459

(57) ABSTRACT

The invention is related to a standoff comprising an acoustic coupling medium for use with an ultrasound transducer, comprising a first portion of tubular shape having a constant cross sectional area with an axis defining the direction of sound propagation and being adapted to be interfaced to the transducer, and a second portion of tapered shape being coaxial to the first portion. The second portion has a proximal end connected to the first, wherein the cross sectional area at a distal end of the second portion is smaller than the cross sectional area at the proximal end. The invention is further related to an envelope which is adapted to enclose the first and/or second portion of the standoff. The invention provides an improved acoustic standoff which is adapted for small and/or superficial structures.

### FIELD OF THE INVENTION

The present invention relates to an acoustic offset or standoff with special requirements for coupling large aperture ultrasound arrays to small targets, in particular therapeutic ultrasound arrays to small targets in pre-clinical and clinical models.

### BACKGROUND OF THE INVENTION

Ultrasound will not propagate for substantial distances through air above a frequency of 100 kHz. As a result, most applications using ultrasound employ an acoustic offset or standoff for visualizing small structures within tissue or within manufactured parts. The standoff consists of an acoustic medium capable of supporting longitudinal waves with minimal acoustic attenuation. These standoffs are placed between the ultrasound transducer and the object to be imaged in such a way to couple the two objects without an air gap. Traditionally, acoustic offsets can consist of a water-like medium such as acoustic coupling gel or water contained within a deformable container. The purpose of the acoustic standoff beyond the coupling of the transducer to the insonified object is to allow for sufficient focusing of a large aperture transducer to allow treatment or imaging of superficial structures.

Therapeutic ultrasound often uses large aperture transducers in order to maximize the acoustic energy focused within the tissue, to minimize the acoustic radiation intensity across non-treated proximal tissues, and to allow treatment of small volumes. Typical sizes of ultrasound transducers are often 8 to 12 cm in size for treating human and small animal organs. The most common means of coupling these transducers to the tissue is with a water path performed by placing the transducer in a large water bath with an acoustic window placed in such a way so that the animal or human is making contact with the acoustic window.

In some experiments, the use of therapeutic transducers encounters the additional difficulty of addressing fairly superficial structures just below the skin line. In case of imaging small animals, smaller organ systems and pathologies enhance these difficulties. These requirements will often drive the design requirements for the transducer to comprise a large aperture and to be aggressively focused. The use of an acoustic standoff therefore becomes a necessity.

Standoffs traditionally have to satisfy several requirements, e.g. positioning the focus and field of view of the ultrasound transducer and to be light-weight. In the case of small animal imaging and therapy, these standoffs must, furthermore, not cause undue distress to the patient or animal through pressure that can lead to breathing difficulty.

US 2006/0058705 A1 discloses geometrically shaped hydrogel standoffs in the shape of a cone for coupling high intensity focused ultrasound for hemostasis and ablation of tissue and blood vessels during surgery. The hydrogel has mechanical and acoustical properties such that the ultrasound transmission standoff members of various dimensions and structural configurations function as ultrasound transmission media and devices within which the ultrasound beam can be transferred to a focal point at the tip of the cone-shaped standoff or in close proximity to it.

### SUMMARY OF THE INVENTION

It would now be desirable to provide an improved acoustic standoff for an ultrasound transducer which is adapted for small and/or superficial structures.

The present invention provides a standoff comprising an acoustic coupling medium for use with an ultrasound transducer, e.g. a therapy or imaging transducer. The standoff comprises a first portion of, e.g. substantially tubular, shape having a substantially constant cross section along the direction of sound propagation and being adapted to be mounted on the transducer. I.e., the shape of the first portion basically corresponds to an upright cylinder with constant but arbitrary cross section. E.g., the cross section may be circular, elliptical or rectangular. Furthermore, the standoff comprises a second portion of tapered shape being coaxial to the first portion and having a proximal end and a distal end, wherein the cross sectional area at the distal end is smaller than the cross sectional area at the proximal end. Therein said proximal end of the second portion is connected to the distal end of the first portion.

In a preferred embodiment of the present invention, the second portion is shaped as to match the acoustic profile of the ultrasound transducer. In other words, the tapered shape or outer surface between distal and proximal end of the second portion of the transducer preferably corresponds to a surface of equal intensity of the ultrasound beam profile, e.g., a −20 dB profile surface. In some embodiments of the present invention, the second portion of the standoff may be cone-shaped, the axis of rotational symmetry being the direction of sound propagation. The outer surface of the second portion between distal and proximal end may as well comprise a curved taper, the curvature being concave or convex. In particular, the angular pitch or curvature of the taper is designed in order to match the transducer beam profile. This shape allows for an optimized focussing of the ultrasound close to the distal end of the standoff, while at the same time minimizing acoustic reflection at the walls of the second portion.

The distal end of the taper or curvature is preferably planar and may comprise an acoustic window of a size that allows coupling of, e.g., a small animal to the standoff. In some embodiments of the present invention the window is rigid.

According to another aspect of the present invention, the first and second portion may have a circular, elliptical or rectangular cross section in a plane perpendicular to the direction of sound propagation.

In a preferred embodiment of the present invention the first and/or second portion consists of an acoustic coupling medium, which can be chosen from at least the following group: acoustic coupling gels, water, water-like media, hydrogels, methylmethacrylates, blends of collagen/poly(acrylic acid), poly vinyl alcohol and the like. This acoustic coupling medium is advantageous for preventing the ultrasound wave from attenuation within the standoff. Therefore, media with a small attenuation coefficient are preferred. First and second portion may also consist of different coupling media.

In a further embodiment the standoff comprises an envelope which tightly encloses the first and/or second portion or parts thereof. The envelope may have a shape substantially identical to the outer surface of first and/or second portion. The envelope may also comprise a first and second portion corresponding to the first and second portion of the standoff. The first and second portion of the envelope may be made of one piece or may be attached to each other. The envelope allows for use of a fluid acoustic coupling medium providing a container for said medium. In this case it may be advantageous to attach the first and second portion of the envelope with a thread and an O-ring therebetween. It is also conceivable to provide an envelope for the first fluid portion only, the second portion consisting of a solid- or gel-like acoustic coupling medium.

In another embodiment, the standoff further comprises a support element adapted to support the transducer, which is arranged at the proximal end of the first portion. Said support may be movable with respect to the second portion along the direction of sound propagation and/or rotatable around the axis defined by the direction of sound propagation by use of a moving means. In order to allow for translations of the support element together with the transducer along the direction of sound propagation it is advantageous if at least the first portion consists of a fluid coupling medium. This enables the user to adjust the ultrasound focus in the direction of sound propagation while the standoff stays in contact with the tissue to be imaged or treated. The envelope is designed to allow for movement of the support element and the transducer and to halt said movement just before reaching a position where the acoustic beam profile would interact with the tapered or curved surface of the second portion of the standoff. A focus adjustment would also be possible in case of solid- or gel-like acoustic coupling media. However, in this case first portions of varying thickness would have to be inserted in order to move the transducer with respect to the tip of the second portion.

The second portion is preferably closed by an acoustic window at the distal end, wherein said acoustic window is substantially transparent for ultrasound. The acoustic window may comprise a gel and/or a gel-like solid. It may as well comprise a membrane made from, e.g., biaxially-oriented polyethylene terephthalate (like Mylar™), a thermoplastic elastomer (like Santoprene™), a plastic wrap or the like. The window may be part of the envelope and ensures that the acoustic coupling medium stays within the standoff while at the same time allowing for basically unhindered ultrasound transmission. However, it may be advantageous if the membrane allows for minor leakage in order to wet the outside of the distal end of the second portion in order to improve coupling between standoff and tissue. Another way to improve this coupling is to provide an acoustic sponge or gel container on the exterior portion of the acoustic window or on the outside of the distal end of the second portion. This as well provides a better acoustic contact between the tissues to be imaged or treated and the standoff. It may be especially helpful in case of rough or uneven tissue surfaces.

In order to monitor the in situ temperature variation due to the application of the acoustic medium, a thermo couple may be provided.

In a preferred embodiment of the present invention, the envelope enclosing the standoff is made of an optically transparent material like acrylic polymers such as polymethyl methacrylate, polycarbonates, polysulfones, polystyrenes, styrene-butadiene copolymers, cellulosis, thermoplastic polyesters and glass to allow for visual inspection of the interior of the standoff. This enables the user of the standoff to ensure that there is no accumulation of bubbles on the surface of the transducer. In some embodiments, the standoff may further comprise a means for gripping the standoff, e.g., a handle or a shaped plastic.

The shape of the acoustic standoff is designed to allow for several advantages: It enables movement of the transducer relative to the second portion of the standoff along the main direction of sound propagation. The cone-like focusing is designed to start at a point where the cone shape substantially matches the acoustic field of the transducer thereby minimizing acoustic reflection and ensuring that the user does not operate the transducer in a region where the beam is not transmitted to the tissue. The size of the acoustic window is chosen to match small structures, e.g. the anatomy of a small animal so that the point of contact with the animal is minimized. This design allows for much more accurate placement of the transducer/standoff pair using external morphological landmarks. In addition, the acoustic window may be designed to allow any imaging transducer placed with a therapy transducer to have an adequate imaging window into the animal. The initial placement of the therapy transducer may also be guided by visual observation (or a laser pointing device) of the targeted region through a therapeutic transducer centre hole and the acoustic window.

The shape of the standoff is also designed so that the amount of acoustic coupling fluid is minimized while still allowing for axial adjustment of the transducer so that superficial structures can be treated. The rigidity of the acoustic window and the standoff itself is a beneficial choice since this will allow removal of all weight of the standoff and transducer from, e.g., the animal. This ensures that the animal will not risk suffocation with insonification proceeds.

These and other aspects of the invention will be apparent from and elucidated with reference to the embodiments described hereinafter.

### BRIEF DESCRIPTION OF THE DRAWINGS

FIG. 1a schematically shows the shape of an acoustic standoff according to a preferred embodiment of the present invention.

FIG. 1b schematically shows a side view of an acoustic standoff with an envelope according to a preferred embodiment of the present invention.

FIG. 2 shows an acoustic profile, i.e. the ultrasound intensity versus location, together with a schematic of a standoff and a transducer according to a preferred embodiment of the present invention.

FIG. 3 shows a photograph of a realization of one embodiment of the present invention.

FIG. 4a schematically shows a top view of a standoff with an envelope according to a preferred embodiment of the present invention that allows for translation of the transducer along the direction of the sound propagation.

FIG. 4b schematically shows a top view of a transducer vertical drive according to a preferred embodiment of the present invention that allows for translation of the transducer along the direction of the sound propagation.

FIG. 5 schematically shows the shape of an acoustic standoff according to a further embodiment of the present invention.

### DETAILED DESCRIPTION OF EMBODIMENTS

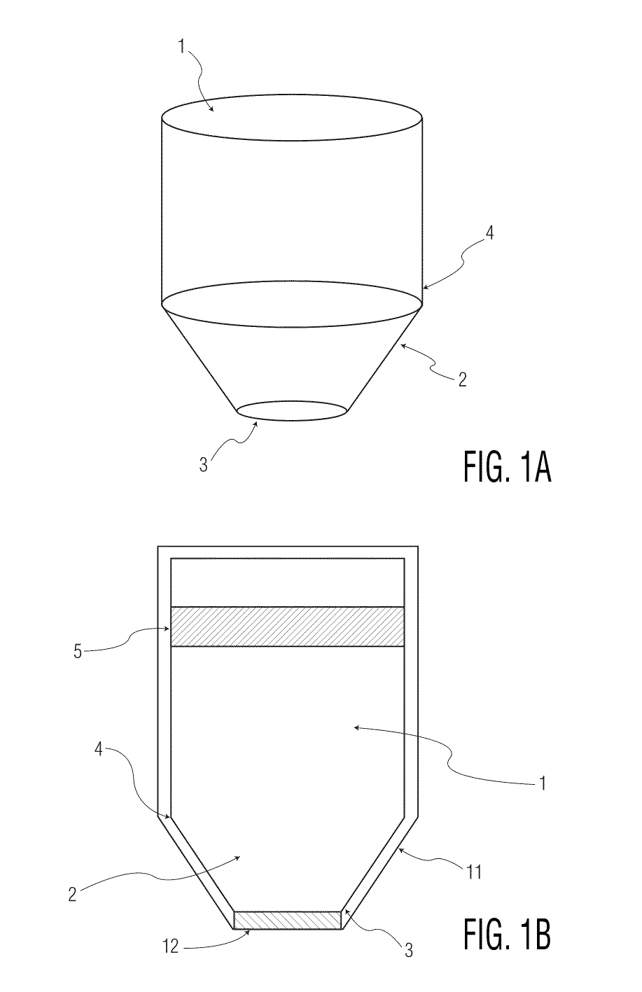

FIG. 1a schematically shows the shape of an acoustic standoff according to a preferred embodiment of the present invention. The standoff comprises a first cylindrical portion 1 and a second cone-shaped portion 2 with a distal end 3 and a proximal end 4. The two portions 1 and 2 are connected to each other at the proximal end 4 of the second portion 2.

FIG. 1b schematically shows a side view of a further embodiment according to the present invention, further comprising an envelope 11 and an acoustic window 12. The diameter of the first portion 1 or rather the respective portion of the envelope is chosen to match the diameter of a transducer 5 with some spacing allowed between the outer diameter of the transducer 5 and the inner diameter of the envelope in order to allow for easy frictionless movement of the transducer with respect to the second portion 2 of the standoff along the main direction of sound propagation., the proximal end 4 being at the same time the end position for translation of the transducer. The envelope may be made of one piece or comprise two portions being attached to each other, said attachment being permanent or releasable. E.g., the two portions of the envelope may be connected to each other via a threaded portion and sealed with an O-ring. The tip of the cone of cone-shaped portion 2 is cut off as to provide a planar distal end 3 for window 12. The first portion 1 and/or second portion 2 may consist of a gel- or solid-like acoustic coupling medium as in FIG. 1a or of a fluid acoustic coupling medium as in FIG. 1b. It is also conceivable that the envelope is thicker and of different shape. E.g., the envelope may further comprise a support means for transducer 5 not shown in FIG. 1b.

The second portion is not restricted to a cone-like shape. Other forms of taper are conceivable as well. E.g., FIG. 5 shows an embodiment with a curved taper, the taper being concave. The cross section of the first and second portion is also not limited to a circle, but may be elliptical, rectangular or of any other shape.

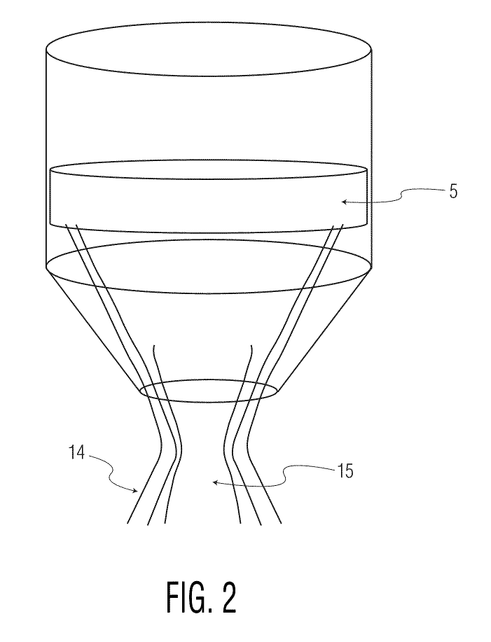

A basic feature of the taper becomes apparent in FIG. 2 showing “equi-intensity” lines 14 indicating lines along which the intensity of the ultrasound beam profile is equal. In order to provide a point of reference, FIG. 2 also shows a schematic of the standoff and transducer which the profile is calculated for. It is apparent from FIG. 2 that the standoff according to the present invention is shaped as to match the acoustic profile of the ultrasound transducer, which is cone-like in this case. In terms of the “equi-intensity” lines, the taper substantially corresponds to the form of these lines. However, slightly above the focus 15 of the ultrasound beam profile, the taper abruptly deviates from these lines forming a planar distal end 3 in order to provide an ultrasound focus 15 close to the distal end, but outside of the standoff.

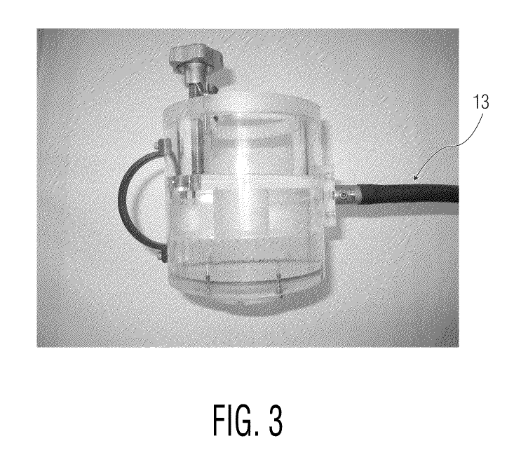

In this case a transducer of a diameter of 8 cm and 8 cm focal length (f number=1) produces ultrasound of 1.2 MHz into a water-like medium, which demands for at least a 45 degree angle for the taper. Ultrasound beams of other acoustic profiles obviously demand for other angular pitches of the taper or other curvatures thereof, respectively. The acoustic window is chosen to occur at a depth of 6.68 cm below the transducer's most distal position, in order to allow focusing at least 1 to 2 cm into the tissue. FIG. 3 shows a photograph of such a device further having a pipe 13 for supplying and/or removing a fluid acoustic coupling medium to/from the standoff.

Another embodiment might replace the tapered piece with a surface that is not described by rotating a straight line around the vertical axis of symmetry. This embodiment may employ a curve that rotated around the same axis of symmetry as shown in FIG. 5. This curve would be chosen to substantially match the therapy transducer's pressure field. Different curves would accommodate the shape of transducers with different (higher) F-numbers or multi-element transducers capable of steering off the vertical axis. In one embodiment, the curve could represent the −40 dB beam profile of the spatial distribution of the acoustic field referenced to the on-axis field.

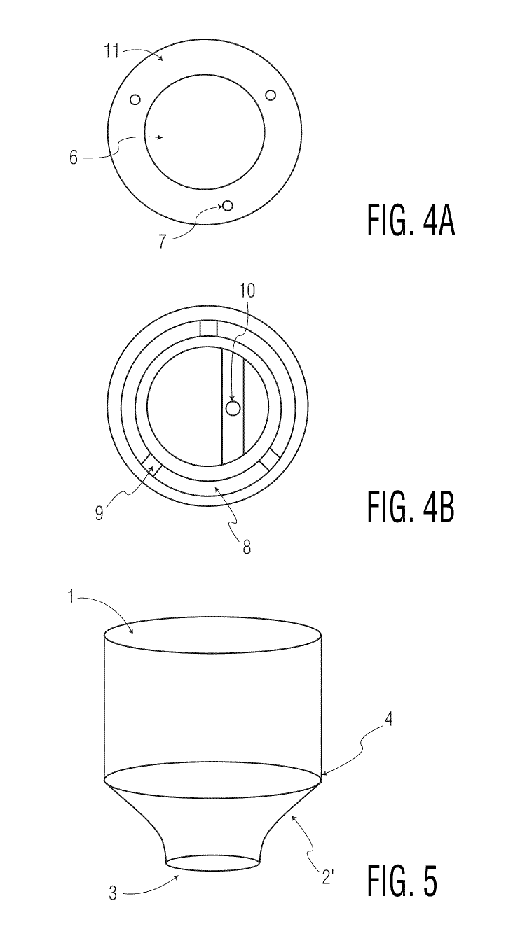

In another embodiment, the standoff may comprise a support element for the transducer, which is adapted to be moved relative to the second portion along the main direction of sound propagation. One conceivable embodiment of such a support element including a translation means is shown in FIG. 4. This means could also allow for rotational movement of the transducer around the main direction of sound propagation. FIG. 4a schematically shows a top view of a standoff according to an embodiment of the present invention with an acoustic coupling medium 6 being present in a centre portion. The standoff further comprises an envelope 11 with three vertical posts 7, which are adapted to guide a translation of the transducer along the main direction of sound propagation.

FIG. 4b schematically shows the corresponding transducer support element comprising a channel 8 to allow for rotation, rotational stops 9 to halt the rotation at predetermined positions and a drive screw 10 in order to provide a given displacement along the (vertical) direction of sound propagation.

While the invention has been illustrated and described in detail in the drawings and foregoing description, such illustration and description are to be considered illustrative or exemplary and not restrictive; the invention is not limited to the disclosed embodiments. Variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. Standoff comprising an acoustic coupling medium for use with an ultrasound transducer (5), comprising
a first portion (1) of a shape having a constant cross section with an axis defining the direction of sound propagation and being adapted to be interfaced to the transducer (5); and
a second portion (2) of tapered shape being coaxial to the first portion (1), the second portion (2) having a proximal end (4) connected to the first portion (1) and having the same cross sectional area as the first portion (1), wherein the cross sectional area at a distal end (3) of the second portion (2) is smaller than the cross sectional area at the proximal end (4).

2. Standoff according to claim 1, wherein the distal end (3) of the second portion (2) is planar.

3. Standoff according to claim 1, wherein the tapered shape of the second portion (2) follows a surface of equal ultrasound intensity of the transducer's beam profile.

4. Standoff according to claim 1, wherein the second portion (2) is cone-shaped.

5. Standoff according to any one of claims 1 to 3, wherein the tapered shape is a curved taper, the curvature being concave or convex.

6. Standoff according to claim 1 wherein the first and second portion have a circular, elliptical or rectangular cross section in a plane perpendicular to their common axis.

7. Standoff according to claim 1, wherein the first and the second portion are integrally formed.

8. Standoff according to claim 1, wherein the first and the second portion are attached to each other.

9. Standoff according to claim 8, wherein the first and the second portion are attached to each other using a thread.

10. Standoff according to claim 1, comprising an envelope (11) tightly enclosing the first and/or second portion of the standoff.

11. Standoff according to claim 10, the envelope comprising two portions attached to each other with a thread.

12. Standoff according to claim 11, the envelope comprising an O-ring between the two portions.

13. Standoff according to claim 1, comprising a support element adapted to support the transducer (5) and arranged at the proximal end of the first portion (1).

14. Standoff according to claim 13, further comprising means for moving the support element relative to the second portion (2) of the standoff along the direction of sound propagation.

15. Standoff according to claim 13, further comprising means for rotating the support element around the axis defined by the direction of sound propagation.

16. Standoff according to claim 1, wherein the first and/or second portion comprises an acoustic coupling medium.

17. Standoff according to claim 1, wherein the acoustic coupling medium can be chosen from at least the following group: acoustic coupling gels, water, water-like media, hydrogels, methylmethacrylates, blends of collagen/poly(acrylic acid), poly vinyl alcohol.

18. Standoff according to claim 1, wherein the acoustic coupling media of the first and second portions are different.

19. Standoff according to claim 1, further comprising an acoustic sponge or gel container at the distal end (3) of the second portion (2).

20. Envelope adapted to enclose the first and/or second portion of the standoff according to claim 1.

21. Envelope according to claim 20, wherein at least part of the envelope is made of an optically transparent material.

22. Envelope according to claim 21, wherein the optically transparent material is polymethyl methacrylate.

23. Envelope according to claim 20, further comprising an acoustic window (12) at the distal end (3) of the second portion (2).

24. Envelope according to claim 23, wherein the acoustic window (12) comprises a gel and/or a gel-like solid.

25. Envelope according to claim 23, wherein the acoustic window (12) comprises a membrane.

26. Envelope according to claim 25, wherein the membrane is made of one of the following materials: biaxially-oriented polyethylene terephthalate like Mylar™, a thermoplastic elastomer like Santoprene™, or a plastic wrap.

27. Envelope according to claim 25, wherein the membrane allows for small amounts of the acoustic coupling medium to leak.

28. Envelope according to claim 20, further comprising a thermo couple.

29. Envelope according to claim 20, further comprising a means for gripping the standoff.

30. Envelope according to claim 29, wherein the means for gripping is a handle or a shaped plastic.

31. Envelope according to claim 20, further comprising a pipe or tube (13) for adding and/or removing fluid coupling medium to/from the standoff.

32. Method for focusing an acoustic profile of an ultrasound transducer (5) with the steps of:
a) providing
a1) a standoff according to claim 1;
b) arranging the transducer (5) at the proximal end of the first portion (1) of the standoff; and
c) adjusting the position of the transducer (5) with respect to the second portion (2) of the standoff in order to focus the ultrasound beam at a desired position close to the distal end (3) of the second portion (2), said position being within the second portion (2) or outside of the distal end (3) of the second portion (2) of the standoff.
